SEQUENCE LISTING (7 sequences)

SEQ ID NO: 1 - DNA, 951 bp, Artificial Sequence
  Synthetic polynucleotide
ttgccatctggttctgacccagctttctctcaaccaaagtctgttttggacgctggtttgacttgtcaaggtgcttctccatcttctgtttctaagccaatcttgttggttccaggtactggtactactggtccacaatctttcgactctaactggattccattgtctactcaattgggttacactccatgttggatctctccaccaccattcatgttgaacgacactcaagttaacactgagtacatggttaacgctatcactgctttgtacgctggttctggtaacaacaagttgccagttttgacttggtctcaaggtggtttggttgctcaatggggtttgactttcttcccatctatcagatctaaggttgacagattgatggctttcgctccagactacaagggtactgttttggctggtccattggacgctttggctgtttctgctccatctgtttggcaacaaactactggttctgctttgactactgctttgagaaacgctggtggtttgactcaaatcgttccaactactaacttgtactctgctactgacgagatcgttcaaccacaagtttctaactctccattggactcttcttacttgttcaacggtaagaacgttcaagctcaagctgtttgtggtccattgttcgttatcgaccatgctggttctttgacttctcaattctcttacgttgttggtagatctgctttgagatctactactggtcaagctagatctgctgactacggtatcactgactgtaacccattgccagctaacgacttgactccagagcaaaaggttgctgctgctgctttgttggctccagctgctgctgctatcgttgctggtccaaagcaaaactgtgagccagacttgatgccatacgctagaccattcgctgttggtaagagaacttgttctggtatcgttactcca

SEQ ID NO: 2 - protein, 317 aa, Candida antarctica [sequence omitted]

SEQ ID NO: 3 - DNA, 978 bp, Artificial Sequence
  Synthetic polynucleotide
ctcgagaaaagagaggctgaagctttgccatctggttctgacccagctttctctcaaccaaagtctgttttggacgctggtttgacttgtcaaggtgcttctccatcttctgtttctaagccaatcttgttggttccaggtactggtactactggtccacaatctttcgactctaactggattccattgtctactcaattgggttacactccatgttggatctctccaccaccattcatgttgaacgacactcaagttaacactgagtacatggttaacgctatcactgctttgtacgctggttctggtaacaacaagttgccagttttgacttggtctcaaggtggtttggttgctcaatggggtttgactttcttcccatctatcagatctaaggttgacagattgatggctttcgctccagactacaagggtactgttttggctggtccattggacgctttggctgtttctgctccatctgtttggcaacaaactactggttctgctttgactactgctttgagaaacgctggtggtttgactcaaatcgttccaactactaacttgtactctgctactgacgagatcgttcaaccacaagtttctaactctccattggactcttcttacttgttcaacggtaagaacgttcaagctcaagctgtttgtggtccattgttcgttatcgaccatgctggttctttgacttctcaattctcttacgttgttggtagatctgctttgagatctactactggtcaagctagatctgctgactacggtatcactgactgtaacccattgccagctaacgacttgactccagagcaaaaggttgctgctgctgctttgttggctccagctgctgctgctatcgttgctggtccaaagcaaaactgtgagccagacttgatgccatacgctagaccattcgctgttggtaagagaacttgttctggtatcgttactccataa

SEQ ID NO: 4 - protein, 325 aa, Artificial Sequence
  Synthetic polypeptide
LEKREAEALPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 5 - DNA, 8959 bp, Artificial Sequence
  Synthetic polynucleotide
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctttgccatctggttctgacccagctttctctcaaccaaagtctgttttggacgctggtttgacttgtcaaggtgcttctccatcttctgtttctaagccaatcttgttggttccaggtactggtactactggtccacaatctttcgactctaactggattccattgtctactcaattgggttacactccatgttggatctctccaccaccattcatgttgaacgacactcaagttaacactgagtacatggttaacgctatcactgctttgtacgctggttctggtaacaacaagttgccagttttgacttggtctcaaggtggtttggttgctcaatggggtttgactttcttcccatctatcagatctaaggttgacagattgatggctttcgctccagactacaagggtactgttttggctggtccattggacgctttggctgtttctgctccatctgtttggcaacaaactactggttctgctttgactactgctttgagaaacgctggtggtttgactcaaatcgttccaactactaacttgtactctgctactgacgagatcgttcaaccacaagtttctaactctccattggactcttcttacttgttcaacggtaagaacgttcaagctcaagctgtttgtggtccattgttcgttatcgaccatgctggttctttgacttctcaattctcttacgttgttggtagatctgctttgagatctactactggtcaagctagatctgctgactacggtatcactgactgtaacccattgccagctaacgacttgactccagagcaaaaggttgctgctgctgctttgttggctccagctgctgctgctatcgttgctggtccaaagcaaaactgtgagccagacttgatgccatacgctagaccattcgctgttggtaagagaacttgttctggtatcgttactccataagcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 6 - DNA, 3922 bp, Artificial Sequence
  Synthetic polynucleotide
cgcattttggcctcaaataaatcttgagcttttggacatagattatatgttctttcttggaagctctttcagctaatagtgaagtgtttcctactaaggatcgcctccaaacgttccaactacgggcggaggttgcaaagaaaacgggtctctcagcgaattgttctcatccatgagtgagtcctctccgtcctttcctcgcgcctggcaataaagcctccttcggaggagctccgtctagagaataattgctgcctttctgactttcggactagcgccaaccgcgaaccacaccaccacaccatcactgtcacccgtcatagttcatccctctctccttataaagcatctaataggttccacaattgtttgccacaaaaatctcttagcatagcccaattgattacgaaattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctttgccatctggttctgacccagctttctctcaaccaaagtctgttttggacgctggtttgacttgtcaaggtgcttctccatcttctgtttctaagccaatcttgttggttccaggtactggtactactggtccacaatctttcgactctaactggattccattgtctactcaattgggttacactccatgttggatctctccaccaccattcatgttgaacgacactcaagttaacactgagtacatggttaacgctatcactgctttgtacgctggttctggtaacaacaagttgccagttttgacttggtctcaaggtggtttggttgctcaatggggtttgactttcttcccatctatcagatctaaggttgacagattgatggctttcgctccagactacaagggtactgttttggctggtccattggacgctttggctgtttctgctccatctgtttggcaacaaactactggttctgctttgactactgctttgagaaacgctggtggtttgactcaaatcgttccaactactaacttgtactctgctactgacgagatcgttcaaccacaagtttctaactctccattggactcttcttacttgttcaacggtaagaacgttcaagctcaagctgtttgtggtccattgttcgttatcgaccatgctggttctttgacttctcaattctcttacgttgttggtagatctgctttgagatctactactggtcaagctagatctgctgactacggtatcactgactgtaacccattgccagctaacgacttgactccagagcaaaaggttgctgctgctgctttgttggctccagctgctgctgctatcgttgctggtccaaagcaaaactgtgagccagacttgatgccatacgctagaccattcgctgttggtaagagaacttgttctggtatcgttactccataagcggccgcatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 7 - DNA, 3922 bp, Artificial Sequence
  Synthetic polynucleotide
cgcattttggcctcaaataaatcttgagcttttggacatagattatatgttctttcttggaagctctttcagctaatagtgaagtgtttcctactaaggatcgcctccaaacgttccaactacgggcggaggttgcaaagaaaacgggtctctcagcgaattgttctcatccatgagtgagtcctctccgtcctttcctcgcgcctggcaataaagcctccttcggaggagctccgtctagagaataattgctgcctttctgactttcggactagcgccaaccgcgaaccacaccaccacaccatcactgtcacccgtcatagttcatccctctctccttataaagcatctaataggttccacaattgtttgccacaaaaatctcttagcatagcccaattgattacgaaattcgaaacgatgagattcccatctatcttcactgctgttttgttcgctgcttcctctgccttggctgctccagttaacaccactactgaggacgagactgctcagatcccagccgaggctgtcattggatactctgacttggagggagatttcgatgttgctgttttgccattctccaactctaccaacaacggtttgttgttcattaacaccaccatcgcttctatcgctgccaaggaggagggtgtctctctcgagaagagagaagctgaagccttgccatctggttctgacccagctttctctcaaccaaagtctgttttggacgctggtttgacttgtcaaggtgcttctccatcttctgtttctaagccaatcttgttggttccaggtactggtactactggtccacaatctttcgactctaactggattccattgtctactcaattgggttacactccatgttggatctctccaccaccattcatgttgaacgacactcaagttaacactgagtacatggttaacgctatcactgctttgtacgctggttctggtaacaacaagttgccagttttgacttggtctcaaggtggtttggttgctcaatggggtttgactttcttcccatctatcagatctaaggttgacagattgatggctttcgctccagactacaagggtactgttttggctggtccattggacgctttggctgtttctgctccatctgtttggcaacaaactactggttctgctttgactactgctttgagaaacgctggtggtttgactcaaatcgttccaactactaacttgtactctgctactgacgagatcgttcaaccacaagtttctaactctccattggactcttcttacttgttcaacggtaagaacgttcaagctcaagctgtttgtggtccattgttcgttatcgaccatgctggttctttgacttctcaattctcttacgttgttggtagatctgctttgagatctactactggtcaagctagatctgctgactacggtatcactgactgtaacccattgccagctaacgacttgactccagagcaaaaggttgctgctgctgctttgttggctccagctgctgctgctatcgttgctggtccaaagcaaaactgtgagccagacttgatgccatacgctagaccattcgctgttggtaagagaacttgttctggtatcgttactccataagcggccgcatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

(19) United States
(12) Patent Application Publication
(10) Pub. No.: US 2011/0183400 A1
(43) Pub. Date: Jul. 28, 2011

(54) PROCESS FOR PRODUCTION OF LIPASES BY GENETIC MODIFICATION OF YEAST

(75) Inventors: Aline Machado de Castro, Rio de Janeiro (BR); Juliana Vaz Bevilaqua, Rio de Janeiro (BR); Denise Maria Guimarães Freire, Rio de Janeiro (BR); Fernando Araripe Goncalves Torres, Brasilia (BR); Lídia Maria Melo Sant'anna, Rio de Janeiro (BR); Melissa Limoeiro Estrada Gutarra, Rio de Janeiro (BR); Caroline Alexandre Barbosa, Rio de Janeiro (BR); Rodrigo Volcan Almeida, Rio de Janeiro (BR); Reginaldo Ramos de Menezes, Rio de Janeiro (BR); Aline Gomes Cunha, Rio de Janeiro (BR)

(73) Assignee: PETROLEO BRASILEIRO S.A.- PETROBRAS, Rio de Janeiro, RJ (BR)

(21) Appl. No.: 12/972,063

(22) Filed: Dec. 17, 2010

(30) Foreign Application Priority Data
Dec. 17, 2009 (BR) PI 0905122-8

(51) Int. Cl. C12N 9/16

(52) U.S. Cl. 435/196

(57) ABSTRACT

The present invention relates to the construction of optimized synthetic lipase gene expression vectors for the high level expression of recombinant lipases in the yeast. The invention provides an enzymatic approach to the industrial processing of by-products resulting from biodiesel production.

### RELATED APPLICATION

This application claims foreign priority from Brazilian Patent application No. PI 0905122-8, filed on Dec. 17, 2009. The teachings of this priority document are hereby incorporated herein by reference in their entirety.

### FIELD OF THE INVENTION

The present invention relates to the construction of optimized synthetic lipase gene expression vectors for the high level expression of recombinant lipases in the yeast Pichia pastoris and their subsequent high yield recovery from yeast. The invention also discloses the genetic manipulation of the yeast Pichia pastoris for the production of recombinant lipases using a submerged fermentation process.

### BACKGROUND OF THE INVENTION

Lipases (triglycerol ester hydrolases—EC 3.1.1.3) are enzymes that catalyse the degradation of fats and oils, releasing fatty acids, diacylglycerols, monoacylglycerols and glycerol. They are effective in various reactions, for example, esterification, transesterification and interesterification in organic solvents. Lipases are found in animal and plant tissues as well as microorganisms, where they have a fundamental role in lipid metabolism. Although pancreatic lipases are often the most studied, lipases of microbial origin are increasingly the focus of industrial research, because they permit large-scale production.

Enzymes in their native form, free enzymes, have been used through the centuries in the food industry, and more recently in the pharmaceutical and chemical industries. Modern techniques of genetic engineering have made possible the large-scale production of these enzymes, as well as modification of their primary structure, for the purpose of changing some of their physico-chemical and biological characteristics. Manipulation by modification of DNA allows enzymes to be prepared on a large scale and tailored for specific purposes.

At present, lipases account for about 5% of the world market for enzymes; however, there is a strong growth trend owing to their vast field of application. These enzymes display great versatility with respect to thermal stability, resistance to organic solvents, specificity, regioselectivity and stereoselectivity, which is why their share of the world market for industrial enzymes is increasing significantly.

The biological function of lipases is primarily the catalysis of the hydrolysis of triglycerides to produce fatty acids and glycerol. However, in conditions in which there is limited water in the medium, most lipases are able to exert their catalytic activity in reactions of alcoholysis and transesterification, which are of great interest for the petroleum industry for the production of biolubricants and biodiesel.

The technique of genetic manipulation has been used extensively for developing the most varied types of enzymatic systems. For example, the International Patent Application, WO 2003/068926, entitled ‘Over-Expression Of Extremozyme Genes In Pseudomonads And Closely Related Bacteria,’ describes in detail the process of genetic manipulation for the generation of recombinant microorganisms.

The yeast P. pastoris has proved to be one of the most powerful systems of eukaryotic expression owing to characteristics such as: expression in high cellular densities, secretion of heterologous proteins and a well-known fermentative production process (reviewed in Daly, R. and Hearn, M. T., J Mol. Recognit. (2005)18(2):119.

### SUMMARY OF THE INVENTION

The present invention relates to the construction of novel synthetic lipase genes expression vectors for high-level expression in the yeast Pichia pastoris. These enzymes are useful for the enzymatic production of biodiesel.

In one aspect, the invention discloses a method of producing recombinant lipase in yeast comprising the steps of optimizing a lipase gene to generate a recombinant lipase gene for yeast expression, inserting said recombinant lipase gene into an expression vector; transforming yeast with the recombinant lipase gene expression vector; and culturing the transformed yeast to express recombinant lipase, wherein the culturing results in the expression of about 12910 U recombinant lipase activity per litre of culture media.

In one aspect, the transforming of the yeast with the expression vector results in the integration of the expression vector into the genome of the host cell.

In another aspect, the culturing produces a maximum yield of about 328 Units (Spectroph.) of lipase activity per litre of media per hour.

The lipase gene can be from Candida antarctica, Thermomyces lanuginosus or Pseudomonas cepacia. The yeast can be Pichia pastoris.

In one embodiment, the recombinant lipase gene has the DNA sequence of SEQ ID NO. 3 and the amino acid sequence of SEQ ID NO. 4. The recombinant yeast can be cultured at 30° C. with stirring at 400 rpm and at a pH of about 6.0.

In one embodiment, the recombinant lipase is secreted efficiently into the culture media.

In another embodiment, the culture media contains glycerin obtained from soya, castor seed, sweet pine-nut, sunflower, macauba, frying oil.

In one embodiment, the glycerin is residual glycerin from the production of biodiesel.

In one embodiment, the culturing in the presence of residual glycerin increases the yield of recombinant lipase as compared to the yield obtained by culturing in the presence of glycerin.

In another embodiment, the culturing in the presence of residual glycerin yields about 18.7 U (Spectroph.) of lipase activity per gram of residual glycerin added to the culture media.

### BRIEF DESCRIPTION OF THE DRAWINGS

FIG. 1 shows the digestion of the vector pBSKLIPB with the restriction enzymes XhoI and NotI. M-λEcoRI/HindIII marker, 1-intact pBSKLIPB, 2-pBSK LIPB digested with the XhoI and NotI enzymes. The arrow indicates the fragment of ˜980 bp corresponding to the LIPB gene.

FIG. 2 shows the physical map of the pPIC9 vector. Taken from the manual Pichia Expression kit (Invitrogen).

FIG. 3 shows the physical map of the pPIC LIPB vector.

FIG. 4 shows the restriction analysis of 3 clones of Escherichia coli transformed with the pPIC_LIPB vector. M1-λEcoRI/HindIII marker, M2-λBstEII marker. i-Intact vector; d-Vector digested with the XhoI and NotI restriction enzymes. The arrows indicate the fragment of the LIPB gene (0.98 kb) and fragment of the pPIC9 vector (8 kb).

FIG. 5 shows the physical map of the pPGKΔ3_LIPB vector.

FIG. 6 shows a restriction analysis of six clones of Escherichia coli transformed with the pPGKΔ3_LIPB vector. M-λEcoRI/HindIII marker. i-intact; d-digested. The arrows indicate fragments corresponding to the LIPB gene (0.98 kb) and to the pPGKΔ3 vector (2.9 kb).

FIG. 7 shows the physical map of the pPGKΔ3_PRO_LIPB vector.

FIG. 8 shows restriction analysis of 6 clones of Escherichia coli transformed with the pPGKΔ3_PRO_LIPB vector. M-λEcoRI/HindIII marker; i-Intact vector; d-Vector digested with the XhoI and NotI restriction enzymes. The arrows indicate fragments corresponding to the LIPB gene (0.98 Kb) and to the pPGKΔ3_PRO vector (3.4 Kb).

FIG. 9 shows the enzymatic plate assay for the clones of P. pastoris transformed with the constitutive expression vectors pPGKΔ3_PRO_LIPB and pPGKΔ3_LIPB which were incubated on plates containing tributyrin for 20 or 40 hours. The arrows indicate the positions of the negative controls.

FIG. 10 shows the enzymatic plate assay for selection of lipase-producing clones with induced expression vector. The arrows indicate the negative controls.

FIG. 11 shows the analysis in SDS-PAGE 12% of the supernatants of Pichia cultures transformed with the pPGKΔ3_PRO_LIPB vector. Clone pPZα-negative control. The arrow indicates the band relating to the lipase CALB. M-marker from Fermentas (Unstained Protein Molecular Weight Marker).

FIG. 12 shows analysis in SDS-PAGE 12% of supernatants of cultures of Pichia transformed with the pPGKΔ3_LIPB vector. Clone pPZα-negative control. The arrow indicates the band relating to lipase CALB. M-marker from Fermentas (Unstained Protein Molecular Weight Marker).

FIG. 13 presents the graph of production of lipases by recombinant P. pastoris using raw soya glycerin as a source of carbon.

FIG. 14 presents the graph of production of lipases by recombinant P. pastoris using raw castor seed glycerin as a source of carbon.

FIG. 15 presents the graph of production of lipases by recombinant P. pastoris using pure glycerin as a source of carbon.

### DETAILED DESCRIPTION OF THE INVENTION

Unless defined otherwise, all technical and scientific terms used herein have the same meaning as is commonly understood by one of ordinary skill in the art. The following definitions are provided to help interpret the disclosure and the claims of this application. In the event a definition in this section is not consistent with definitions elsewhere, the definitions set forth in this section will control.

As used herein, the yeast Pichia sp. of the invention may include, but is not limited to, Pichia pastoris, Pichia flnlandica, Pichia trehalophila, Pichia koclamae, Pichia membranaefaciens, Pichia methanolica, Pichia minuta (Ogataea minuta, Pichia lindneri), Pichia opuntiae, Pichia thermotolerans, Pichi salictaria, Pichia guercum, Pichia pijperi, Pichia stiptis.

As used herein, the term “transformed” as known in the art, is the directed modification of an organism's genome or episome via the introduction of external DNA or RNA, or to any other stable introduction of external DNA or RNA.

As is understood in the art, DNA may be transformed into a host cell by several different methods. In yeast, any convenient method of DNA transfer may be used, such as electroporation, the lithium chloride method, or the spheroplast method. To produce a stable strain suitable for high-density fermentation, it is desirable to integrate the DNA into the host chromosome. Integration occurs via homologous recombination, using techniques known in the art. For example, DNA capable of expressing at least one heterologous protein can be provided with flanking sequences homologous to sequences of the host organism. In this manner, integration occurs at a defined site in the host genome, without disruption of desirable or essential genes. Alternatively, DNA capable of expressing at least one heterologous protein is integrated into the site of an undesired gene in a host chromosome, effecting the disruption or deletion of the gene or expression of that gene product. In other embodiments, DNA may be introduced into the host via a chromosome, plasmid, retroviral vector, or random integration into the host genome.

Features and advantages of the present application will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the application covers all modifications, equivalents, and alternatives falling within the spirit and scope of the application as defined by the claims.

Synthesis of the LipB Gene

The gene of lipase B (CALB) C. antarctica, described by Uppenberg et al. (1994) Structure 15; 2(4): 293-308, codes for a protein of 317 amino acid residues with molecular mass of 33273 Dalton.

The lipase CalB is made in the form of a pre-protein that is processed proteolytically in the endoplasmic reticulum for removal of the signal peptide and “pro” region before being secreted. In this non-limiting example only the version that encodes the mature version of the enzyme is synthesized chemically since the intrinsic secretion signals of P. pastoris will be used for optimizing gene expression.

Prior to synthesis of the gene that encodes lipase B of C. antarctica a sequence optimization was carried out. The following criteria were taken into consideration in this optimization:

preferential codons of the genes most expressed in P. pastoris,

content of G+C around 50%,

removal of possible secondary structures and cryptic splice sites,

removal of unwanted restriction sites,

addition of restriction sites at the ends of the synthetic gene to facilitate cloning into the expression vector of P. pastoris.

introduction of a His6 tag at the C-terminal of the protein to facilitate purification on Ni-NTA affinity columns.

The primary protein sequence of the mature version of lipase CalB (SEQ ID NO. 1) is presented, below:

SEQ ID No. 1:
LPSGSDPAFSQPKSVLDAGLTCQGASPSSVSKPILLVPGTGTTGPQSFDS
NWIPLSTQLGYTPCWISPPPFMLNDTQVNTEYMVNAITALYAGSGNNKLP
VLTWSQGGLVAQWGLTFFPSIRSKVDRLMAFAPDYKGTVLAGPLDALAVS
APSVWQQTTGSALTTALRNAGGLTQIVPTTNLYSATDEIVQPQVSNSPLD
SSYLFNGKNVQAQAVCGPLFVIDRAGSLTSQFSYVVGRSALRSTTGQARS
ADYGITDCNPLPKNDLTPEQKVAAAALLAPAAAAIVAGPKQNCEPDLMPY
ARPFAVGKRTCSGIVTP

This protein sequence is encoded by the native gene sequence of C. antarctica (gene CALB; SEQ ID NO. 2) shown below.

SEQ ID NO. 2
CTACCTTCCGGTTCGGACCCTGCCTTTTCGCAGCCCAAGTCGGTGCTCGA
TGCGGGTCTGACCTGCCAGGGTGCTTCGCCATCCTCGGTCTCCAAACCCA
TCCTTCTCGTCCCCGGAACCGGCACCACAGGTCCACAGTCGTTCGACTCG
AACTGGATCCCCCTCTCAACGCAGTTGGGTTACACACCCTGCTGGATCTC
ACCCCCGCCGTTCATGCTCAACGACACCCAGGTCAACACGGAGTACATGG
TCAACGCCATCACCGCGCTCTACGCTGGTTCGGGCAACAACAAGCTTCCC
GTGCTTACCTGGTCCCAGGGTGGTCTGGTTGCACAGTGGGGTCTGACCTT
CTTCCCCAGTATCAGGTCCAAGGTCGATCGACTTATGGCCTTTGCGCCCG
ACTACAAGGGCACCGTCCTCGCCGGCCCTCTCGATGCACTCGCGGTTAGT
GCACCCTCCGTATGGCAGCAAACCACCGGTTCGGCACTCACCACCGCACT
CCGAAACGCAGGTGGTCTGACCCAGATCGTGCCCACCACCAACCTCTACT
CGGCGACCGACGAGATCGTTCAGCCTCAGGTGTCCAACTCGCCACTCGAC
TCATCCTACCTCTTCAACGGAAAGAACGTCCAGGCACAGGCCGTGTGTGG
GCCGCTGTTCGTCATCGACCATGCAGGCTCGCTCACCTCGCAGTTCTCCT
ACGTCGTCGGTCGATCCGCCCTGCGCTCCACCACGGGCCAGGCTCGTAGT
GCAGACTATGGCATTACGGACTGCAACCCTCTTCCCGCCAATGATCTGAC
TCCCGAGCAAAAGGTCGCCGCGGCTGCGCTCCTGGCGCCGGCAGCTGCAG
CCATCGTGGCGGGTCCAAAGCAGAACTGCGAGCCCGACCTCATGCCCTAC
GCCCGCCCCTTTGCAGTAGGCAAAAGGACCTGCTCCGGCATCGTCACCCC
C

The optimized DNA sequence of the gene LipB (SEQ ID NO. 3) is depicted below:

SEQ ID NO. 3
TTGCCATCTGGTTCTGACCCAGCTTTCTCTCAACCAAAGTCTGTTTTGGA
CGCTGGTTTGACTTGTCAAGGTGCTTCTCCATCTTCTGTTTCTAAGCCAA
TCTTGTTGGTTCCAGGTACTGGTACTACTGGTCCACAATCTTTCGACTCT
AACTGGATTCCATTGTCTACTCAATTGGGTTACACTCCATGTTGGATCTC
TCCACCACCATTCATGTTGAACGACACTCAAGTTAACACTGAGTACATGG
TTAACGCTATCACTGCTTTGTACGCTGGTTCTGGTAACAACAAGTTGCCA
GTTTTGACTTGGTCTCAAGGTGGTTTGGTTGCTCAATGGGGTTTGACTTT
CTTCCCATCTATCAGATCTAAGGTTGACAGATTGATGGCTTTCGCTCCAG
ACTACAAGGGTACTGTTTTGGCTGGTCCATTGGACGCTTTGGCTGTTTCT
GCTCCATCTGTTTGGCAACAAACTACTGGTTCTGCTTTGACTACTGCTTT
GAGAAACGCTGGTGGTTTGACTCAAATCGTTCCAACTACTAACTTGTACT
CTGCTACTGACGAGATCGTTCAACCACAAGTTTCTAACTCTCCATTGGAC
TCTTCTTACTTGTTCAACGGTAAGAACGTTCAAGCTCAAGCTGTTTGTGG
TCCATTGTTCGTTATCGACCATGCTGGTTCTTTGACTTCTCAATTCTCTT
ACGTTGTTGGTAGATCTGCTTTGAGATCTACTACTGGTCAAGCTAGATCT
GCTGACTACGGTATCACTGACTGTAACCCATTGCCAGCTAACGACTTGAC
TCCAGAGCAAAAGGTTGCTGCTGCTGCTTTGTTGGCTCCAGCTGCTGCTG
CTATCGTTGCTGGTCCAAAGCAAAACTGTGAGCCAGACTTGATGCCATAC
GCTAGACCATTCGCTGTTGGTAAGAGAACTTGTTCTGGTATCGTTACTCC
A

The optimized sequence had the following parameters:

Content of G+C, 45.4%

Codon usage:

| | | | | | | | | | | | | | | | |
|---|---|---|---|---|---|---|---|---|---|---|---|---|---|---|---|
| Phe | UUU | 0 | 0.00 | Ser | UCU | 31 | 6.00 | Tyr | UAU | 0 | 0.00 | Cys | UGU | 6 | 2.00 |
| | UUC | 10 | 2.00 | | UCC | 0 | 0.00 | | UAC | 9 | 2.00 | | UGC | 0 | 0.00 |

| | | | | | | | | | | | | | | |
|---|---|---|---|---|---|---|---|---|---|---|---|---|---|---|
| Leu | UUA | 0 | 0.00 | UCA | 0 | 0.00 | TER | UAA | 1 | 3.00 | TER | UGA | 0 | 0.00 |

| | | | | | | | | | | | | | | | |
|---|---|---|---|---|---|---|---|---|---|---|---|---|---|---|---|
| | UUG | 31 | 6.00 | | UCG | 0 | 0.00 | | UAG | 0 | 0.00 | Trp | UGG | 5 | 1.00 |
| | CUU | 0 | 0.00 | Pro | CCU | 0 | 0.00 | His | CAU | 1 | 2.00 | Arg | CGU | 0 | 0.00 |
| | CUC | 0 | 0.00 | | CCC | 0 | 0.00 | | CAC | 0 | 0.00 | | CGC | 0 | 0.00 |
| | CUA | 0 | 0.00 | | CCA | 30 | 4.00 | Gln | CAA | 18 | 2.00 | | CGA | 0 | 0.00 |
| | CUG | 0 | 0.00 | | CCG | 0 | 0.00 | | CAG | 0 | 0.00 | | CGG | 0 | 0.00 |
| Ile | AUU | 1 | 0.27 | Thr | ACU | 27 | 4.00 | Asn | AAU | 0 | 0.00 | Ser | AGU | 0 | 0.00 |
| | AUC | 10 | 2.73 | | ACC | 0 | 0.00 | | AAC | 14 | 2.00 | | AGC | 0 | 0.00 |
| | AUA | 0 | 0.00 | | ACA | 0 | 0.00 | Lys | AAA | 0 | 0.00 | Arg | AGA | 8 | 6.00 |
| Met | AUG | 4 | 1.00 | | ACG | 0 | 0.00 | | AAG | 9 | 2.00 | | AGG | 0 | 0.00 |
| Val | GUU | 23 | 4.00 | Ala | GCU | 36 | 4.00 | Asp | GAU | 0 | 0.00 | Gly | GGU | 26 | 4.00 |
| | GUC | 0 | 0.00 | | GCC | 0 | 0.00 | | GAC | 14 | 2.00 | | GGC | 0 | 0.00 |
| | GUA | 0 | 0.00 | | GCA | 0 | 0.00 | Glu | GAA | 0 | 0.00 | | GGA | 0 | 0.00 |
| | GUG | 0 | 0.00 | | GCG | 0 | 0.00 | | GAG | 4 | 2.00 | | GGG | 0 | 0.00 |

Translation of the optimized DNA sequence of the gene LipB (SEQ ID NO. 3) is depicted below (Amino acid sequence on top line is SEQ ID NO: 4; DNA sequence below amino acid sequence is SEQ ID NO: 3):

| | |
|---|---|
| 1 | L P S G S D P A F S Q P K S V L D A G L |
| 1 | TTGCCATCTGGTTCTGACCCAGCTTTCTCTCAACCAAAGTCTGTTTTGGACGCTGGTTTG |
| 21 | T C Q G A S P S S V S K P I L L V P G T |
| 61 | ACTTGTCAAGGTGCTTCTCCATCTTCTGTTTCTAAGCCAATCTTGTTGGTTCCAGGTACT |
| 41 | G T T G P Q S F D S N W I P L S T Q L G |
| 121 | GGTACTACTGGTCCACAATCTTTCGACTCTAACTGGATTCCATTGTCTACTCAATTGGGT |
| 61 | Y T P C W I S P P P F M L N D T Q V N T |
| 181 | TACACTCCATGTTGGATCTCTCCACCACCATTCATGTTGAACGACACTCAAGTTAACACT |
| 81 | E Y M V N A I T A L Y A G S G N N K L P |
| 241 | GAGTACATGGTTAACGCTATCACTGCTTTGTACGCTGGTTCTGGTAACAACAAGTTGCCA |
| 101 | V L T W S Q G G L V A Q W G L T F F P S |
| 301 | GTTTTGACTTGGTCTCAAGGTGGTTTGGTTGCTCAATGGGGTTTGACTTTCTTCCCATCT |
| 121 | I R S K V D R L M A F A P D Y K G T V L |
| 361 | ATCAGATCTAAGGTTGACAGATTGATGGCTTTCGCTCCAGACTACAAGGGTACTGTTTTG |
| 141 | A G P L D A L A V S A P S V W Q Q T T G |
| 421 | GCTGGTCCATTGGACGCTTTGGCTGTTTCTGCTCCATCTGTTTGGCAACAAACTACTGGT |
| 161 | S A L T T A L R N A G G L T Q I V P T T |
| 481 | TCTGCTTTGACTACTGCTTTGAGAAACGCTGGTGGTTTGACTCAAATCGTTCCAACTACT |
| 181 | N L Y S A T D E I V Q P Q V S N S P L D |
| 541 | AACTTGTACTCTGCTACTGACGAGATCGTTCAACCACAAGTTTCTAACTCTCCATTGGAC |
| 201 | S S Y L F N G K N V Q A Q A V C G P L F |
| 601 | TCTTCTTACTTGTTCAACGGTAAGAACGTTCAAGCTCAAGCTGTTTGTGGTCCATTGTTC |
| 221 | V I D H A G S L T S Q F S Y V V G R S A |
| 661 | GTTATCGACCATGCTGGTTCTTTGACTTCTCAATTCTCTTACGTTGTTGGTAGATCTGCT |
| 241 | L R S T T G Q A R S A D Y G I T D C N P |
| 721 | TTGAGATCTACTACTGGTCAAGCTAGATCTGCTGACTACGGTATCACTGACTGTAACCCA |
| 261 | L P A N D L T P E Q K V A A A A L L A P |
| 781 | TTGCCAGCTAACGACTTGACTCCAGAGCAAAAGGTTGCTGCTGCTGCTTTGTTGGCTCCA |
| 281 | A A A A I V A G P K Q N C E P D L M P Y |
| 841 | GCTGCTGCTGCTATCGTTGCTGGTCCAAAGCAAAACTGTGAGCCAGACTTGATGCCATAC |
| 301 | A R P F A V G K R T C S G I V T P |
| 901 | GCTAGACCATTCGCTGTTGGTAAGAGAACTTGTTCTGGTATCGTTACTCCA |

Alignment of the gene CALB (wild-type; SEQ ID NO: 1)×LipB (optimized; SEQ ID NO: 3) using the software CLUSTAL 2.0.1 Multiple Sequence Alignment showed that about 8% of the nucleotides were modified in the optimized version:

| | | |
|---|---|---|
| CALB | CTACCTTCCGGTTCGGACCCTGCCTTTTCGCAGCCCAAGTCGGTGCTCGATGCGGGTCTG | 60 |
| LipB | TTGCCATCTGGTTCTGACCCAGCTTTCTCTCAACCAAAGTCTGTTTTGGACGCTGGTTTG | 60 |
| | * ** ** ***** ***** ** ** ** ** ** ***** ** * ** ** *** ** | |
| CALB | ACCTGCCAGGGTGCTTCGCCATCCTCGGTCTCCAAACCCATCCTTCTCGTCCCCGGAACC | 120 |
| LipB | ACTTGTCAAGGTGCTTCTCCATCTTCTGTTTCTAAGCCAATCTTGTTGGTTCCAGGTACT | 120 |
| | ** ** ** ******** ***** ** ** ** ** ** *** * * ** ** ** ** | |
| CALB | GGCACCACAGGTCCACAGTCGTTCGACTCGAACTGGATTCCCCTCTCAACGCAGTTGGGT | 180 |
| LipB | GGTACTACTGGTCCACAATCTTTCGACTCTAACTGGATCCCATTGTCTACTCAATTGGGT | 180 |
| | ** ** ** ******** ** ******** ******** ** * ** ** ** ****** | |
| CALB | TACACACCCTGCTGGATCTCACCCCCGCCGTTCATGCTCAACGACACCCAGGTCAACACG | 240 |
| LipB | TACACTCCATGTTGGATCTCTCCACCACCATTCATGTTGAACGACACTCAAGTTAACACT | 240 |
| | ***** ** ** ******** ** ** ** ****** * ******** ** ** ***** | |
| CALB | GAGTACATGGTCAACGCCATCACCGCGCTCTACGCTGGTTCGGGCAACAACAAGCTTCCC | 300 |
| LipB | GAGTACATGGTTAACGCTATCACTGCTTTGTACGCTGGTTCTGGTAACAACAAGTTGCCA | 300 |
| | *********** ***** ***** ** * *********** ** ********* * ** | |
| CALB | GTGCTTACCTGGTCCCAGGGTGGTCTGGTTGCACAGTGGGGTCTGACCTTCTTCCCCAGT | 360 |
| LipB | GTTTTGACTTGGTCTCAAGGTGGTTTGGTTGCTCAATGGGGTTTGACTTTCTTCCCATCT | 360 |
| | ** * ** ***** ** ****** ******* ** ****** **** ******** * | |
| CALB | ATCAGGTCCAAGGTCGATCGACTTATGGCCTTTGCGCCCGACTACAAGGGCACCGTCCTC | 420 |
| LipB | ATCAGATCTAAGGTTGACAGATTGATGGCTTTCGCTCCAGACTACAAGGGTACTGTTTTG | 420 |
| | ***** ** ***** ** ** * ***** ** ** ** *********** ** ** * | |
| CALB | GCCGGCCCTCTCGATGCACTCGCGGTTAGTGCACCCTCCGTATGGCAGCAAACCACCGGT | 480 |
| LipB | GCTGGTCCATTGGACGCTTTGGCTGTTTCTGCTCCATCTGTTTGGCAACAAACTACTGGT | 480 |
| | ** ** ** * ** ** * ** *** *** ** ** ** ***** ***** ** *** | |
| CALB | TCGGCACTCACCACCGCACTCCGAAACGCAGGTGGTCTGACCCAGATCGTGCCCACCACC | 540 |
| LipB | TCTGCTTTGACTACTGCTTTGAGAAACGCTGGTGGTTTGACTCAAATCGTTCCAACTACT | 540 |
| | ** ** * ** ** ** * ******* ****** **** ** ***** ** ** ** | |
| CALB | AACCTCTACTCGGCGACCGACGAGATCGTTCAGCCTCAGGTGTCCAACTCGCCACTCGAC | 600 |
| LipB | AACTTGTACTCTGCTACTGACGAGATCGTTCAACCACAAGTTTCTAACTCTCCATTGGAC | 600 |
| | *** * ***** ** ** ************** ** ** ** ** ***** *** * *** | |
| CALB | TCATCCTACCTCTTCAACGGAAAGAACGTCCAGGCACAGGCCGTGTGTGGGCCGCTGTTC | 660 |
| LipB | TCTTCTTACTTGTTCAACGGTAAGAACGTTCAAGCTCAAGCTGTTTGTGGTCCATTGTTC | 660 |
| | ** ** *** * ******** ******** ** ** ** ** ** ***** ** ***** | |
| CALB | GTCATCGACCATGCAGGCTCGCTCACCTCGCAGTTCTCCTACGTCGTCGGTCGATCCGCC | 720 |
| LipB | GTTATCGACCATGCTGGTTCTTTGACTTCTCAATTCTCTTACGTTGTTGGTAGATCTGCT | 720 |
| | ** *********** ** ** * ** ** ** ***** ***** ** *** **** ** | |
| CALB | CTGCGCTCCACCACGGGCCAGGCTCGTAGTGCAGACTATGGCATTACGGACTGCAACCCT | 780 |
| LipB | TTGAGATCTACTACTGGTCAAGCTAGATCTGCTGACTACGGTATCACTGACTGTAACCCA | 780 |
| | ** * ** ** ** ** ** *** * *** ***** ** ** ** ***** ***** | |
| CALB | CTTCCCGCCAATGATCTGACTCCCGAGCAAAAGGTCGCCGCGGCTGCGCTCCTGGCGCCG | 840 |
| LipB | TTGCCAGCTAACGACTTGACTCCAGAGCAAAAGGTTGCTGCTGCTGCTTTGTTGGCTCCA | 840 |
| | * ** ** ** ** ******* *********** ** ** ***** * **** ** | |
| CALB | GCAGCTGCAGCCATCGTGGCGGGTCCAAAGCAGAACTGCGAGCCCGACCTCATGCCCTAC | 900 |
| LipB | GCTGCTGCTGCTATCGTTGCTGGTCCAAAGCAAAACTGTGAGCCAGACTTGATGCCATAC | 900 |
| | ** ***** ** ***** ** *********** ***** ***** *** * ***** *** | |
| CALB | GCCCGCCCCTTTGCAGTAGGCAAAAGGACCTGCTCCGGCATCGTCACCCCC | 951 |
| LipB | GCTAGACCATTCGCTGTTGGTAAGAGAACTTGTTCTGGTATCGTTACTCCA | 951 |
| | ** * ** ** ** ** ** ** ** ** ** ** ** ***** ** ** | |

Besides the optimizations described above that facilitate cloning into the expression vector of P. pastoris, the following sequences were added to generate SEQ ID NO. 5 shown below: In the 5′ portion of the gene, and in phase with the gene LipB, a sequence corresponding to the site of KEX2 and STE13 of Saccharomyces cerevisiae and a site for XhoI (underlined in the 5′ region of the gene), the stop codon TAA, a His₆ₓ tag to facilitate purification by nickel-NTA affinity chromatography, and a restriction site for the enzyme NotI will be added in the 3′ portion. (Underlined in the 3′ region of the gene)

SEQ ID NO. 5:

CTCGAGAAGAGAGAAGCTGAAGCCTTGCCATCTGGTTCTGACCCAGCTTT
CTCTCAACCAAAGTCTGTTTTGGACGCTGGTTTGACTTGTCAAGGTGCTT
CTCCATCTTCTGTTTCTAAGCCAATCTTGTTGGTTCCAGGTACTGGTACT
ACTGGTCCACAATCTTTCGACTCTAACTGGATTCCATTGTCTACTCAATT
GGGTTACACTCCATGTTGGATCTCTCCACCACCATTCATGTTGAACGACA
CTCAAGTTAACACTGAGTACATGGTTAACGCTATCACTGCTTTGTACGCT
GGTTCTGGTAACAACAAGTTGCCAGTTTTGACTTGGTCTCAAGGTGGTTT
GGTTGCTCAATGGGGTTTGACTTTCTTCCCATCTATCAGATCTAAGGTTG
ACAGATTGATGGCTTTCGCTCCAGACTACAAGGGTACTGTTTTGGCTGGT
CCATTGGACGCTTTGGCTGTTTCTGCTCCATCTGTTTGGCAACAAACTAC
TGGTTCTGCTTTGACTACTGCTTTGAGAAACGCTGGTGGTTTGACTCAAA
TCGTTCCAACTACTAACTTGTACTCTGCTACTGACGAGATCGTTCAACCA
CAAGTTTCTAACTCTCCATTGGACTCTTCTTACTTGTTCAACGGTAAGAA
CGTTCAAGCTCAAGCTGTTTGTGGTCCATTGTTCGTTATCGACCATGCTG
GTTCTTTGACTTCTCAATTCTCTTACGTTGTTGGTAGATCTGCTTTGAGA
TCTACTACTGGTCAAGCTAGATCTGCTGACTACGGTATCACTGACTGTAA
CCCATTGCCAGCTAACGACTTGACTCCAGAGCAAAAGGTTGCTGCTGCTG
CTTTGTTGGCTCCAGCTGCTGCTGCTATCGTTGCTGGTCCAAAGCAAAAC
TGTGAGCCAGACTTGATGCCATACGCTAGACCATTCGCTGTTGGTAAGAG
AACTTGTTCTGGTATCGTTACTCCACATCATCATCATCATCATCATTAAG
CGGCCGC

The translation of the final optimized version of the LipB gene (SE ID NO: 6) is:

| | |
|---|---|
| | KEX2 STE13 STE13 |
| 1 | L E K R E A E A L P S G S D P A F S Q P |
| 1 | CTCGAGAAGAGAGAAGCTGAAGCCTTGCCATCTGGTTCTGACCCAGCTTTCTCTCAACCA |
| | XhoI |
| 21 | K S V L D A G L T C Q G A S P S S V S K |
| 61 | AAGTCTGTTTTGGACGCTGGTTTGACTTGTCAAGGTGCTTCTCCATCTTCTGTTTCTAAG |
| 41 | P I L L V P G T G T T G P Q S F D S N W |
| 121 | CCAATCTTGTTGGTTCCAGGTACTGGTACTACTGGTCCACAATCTTTCGACTCTAACTGG |
| 61 | I P L S T Q L G Y T P C W I S P P P F M |
| 181 | ATTCCATTGTCTACTCAATTGGGTTACACTCCATGTTGGATCTCTCCACCACCATTCATG |
| 81 | L N D T Q V N T E Y M V N A I T A L Y A |
| 241 | TTGAACGACACTCAAGTTAACACTGAGTACATGGTTAACGCTATCACTGCTTTGTACGCT |
| 101 | G S G N N K L P V L T W S Q G G L V A Q |
| 301 | GGTTCTGGTAACAACAAGTTGCCAGTTTTGACTTGGTCTCAAGGTGGTTTGGTTGCTCAA |
| 121 | W G L T F F P S I R S K V D R L M A F A |
| 361 | TGGGGTTTGACTTTCTTCCCATCTATCAGATCTAAGGTTGACAGATTGATGGCTTTCGCT |
| 141 | P D Y K G T V L A G P L D A L A V S A P |
| 421 | CCAGACTACAAGGGTACTGTTTTGGCTGGTCCATTGGACGCTTTGGCTGTTTCTGCTCCA |
| 161 | S V W Q Q T T G S A L T T A L R N A G G |
| 481 | TCTGTTTGGCAACAAACTACTGGTTCTGCTTTGACTACTGCTTTGAGAAACGCTGGTGGT |
| 181 | L T Q I V P T T N L Y S A T D E I V Q P |
| 541 | TTGACTCAAATCGTTCCAACTACTAACTTGTACTCTGCTACTGACGAGATCGTTCAACCA |
| 201 | Q V S N S P L D S S Y L F N G K N V Q A |
| 601 | CAAGTTTCTAACTCTCCATTGGACTCTTCTTACTTGTTCAACGGTAAGAACGTTCAAGCT |
| 221 | Q A V C G P L F V I D H A G S L T S Q F |
| 661 | CAAGCTGTTTGTGGTCCATTGTTCGTTATCGACCATGCTGGTTCTTTGACTTCTCAATTC |
| 241 | S Y V V G R S A L R S T T G Q A R S A D |
| 721 | TCTTACGTTGTTGGTAGATCTGCTTTGAGATCTACTACTGGTCAAGCTAGATCTGCTGAC |
| 261 | Y G I T D C N P L P A N D L T P E Q K V |
| 781 | TACGGTATCACTGACTGTAACCCATTGCCAGCTAACGACTTGACTCCAGAGCAAAAGGTT |
| 281 | A A A A L L A P A A A A I V A G P K Q N |
| 841 | GCTGCTGCTGCTTTGTTGGCTCCAGCTGCTGCTGCTATCGTTGCTGGTCCAAAGCAAAAC |
| 301 | C E P D L M P Y A R P F A V G K R T C S |
| 901 | TGTGAGCCAGACTTGATGCCATACGCTAGACCATTCGCTGTTGGTAAGAGAACTTGTTCT |
| 321 | G I V T P H H H H H H - |
| 961 | GGTATCGTTACTCCACATCATCATCATCATCATTAAGCGGCCGC |
| | His6x tag NotI |

After this complete optimization process, synthesis of the gene, called LipB, was performed by the company Epoch Biolabs (USA).

Construction of Vectors for Expression of the Gene LipB in Pichia pastoris

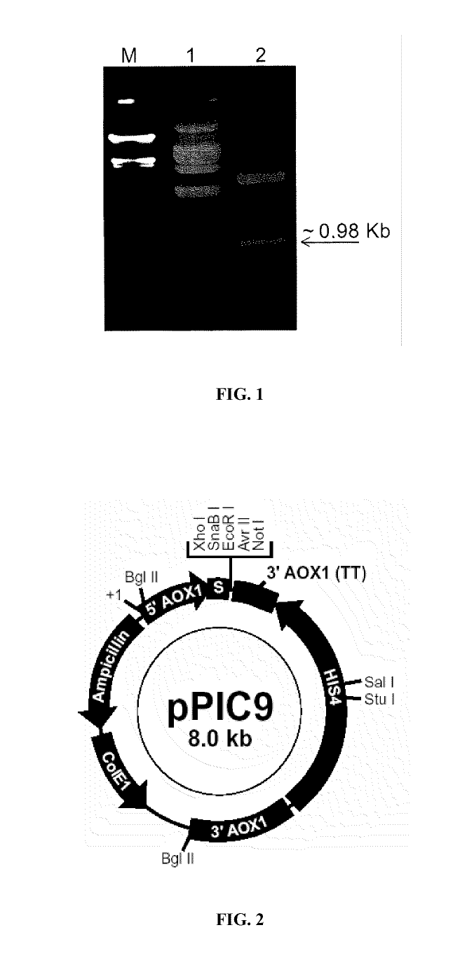

The LipB gene (optimized version of the CALB gene of Candida antarctica) synthesized chemically by the company Epoch Biolabs (USA) was cloned into the pBluescript II SK vector, resulting in the vector pBSK LIPB. This vector was used for transforming thermo-competent Escherichia coli XL10-Gold for amplification and maintenance. For the subcloning of the LipB gene into the expression vector of Pichia, the vector was cleaved with the restriction enzymes XhoI and NotI and the digestion product was resolved on a 0.8% agarose gel. The fragment corresponding to the LipB gene (˜980 bp) (FIG. 1) was eluted from the gel using the QIAquick Gel Extraction kit, according to the manufacturer's specifications.

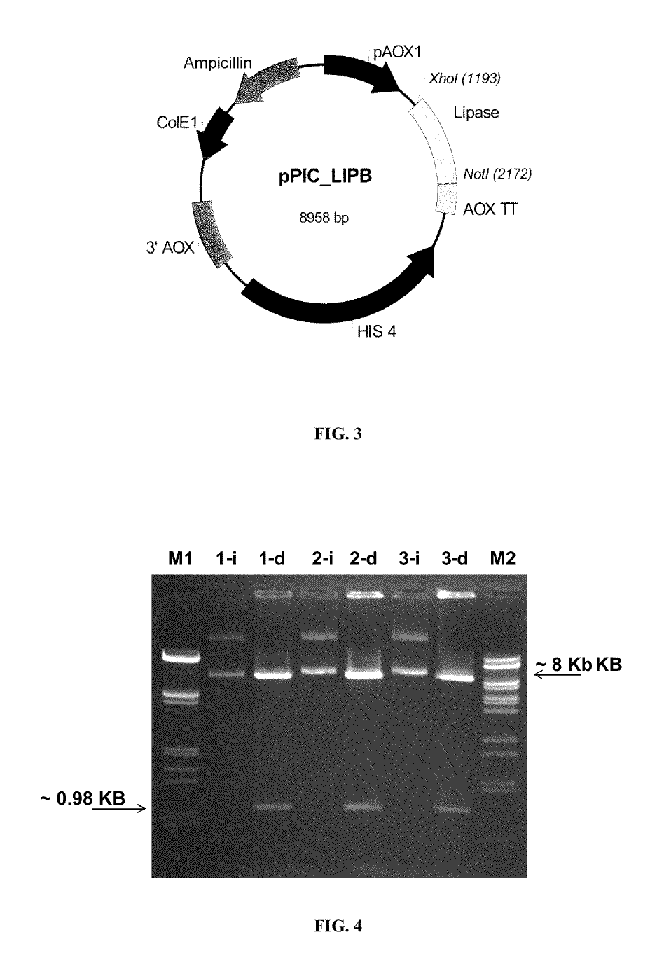

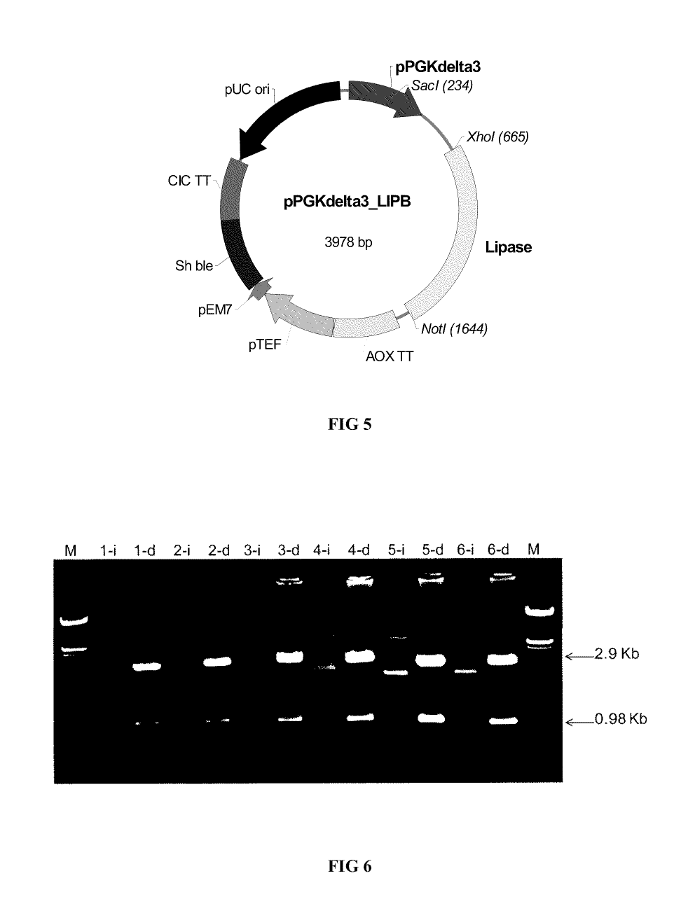

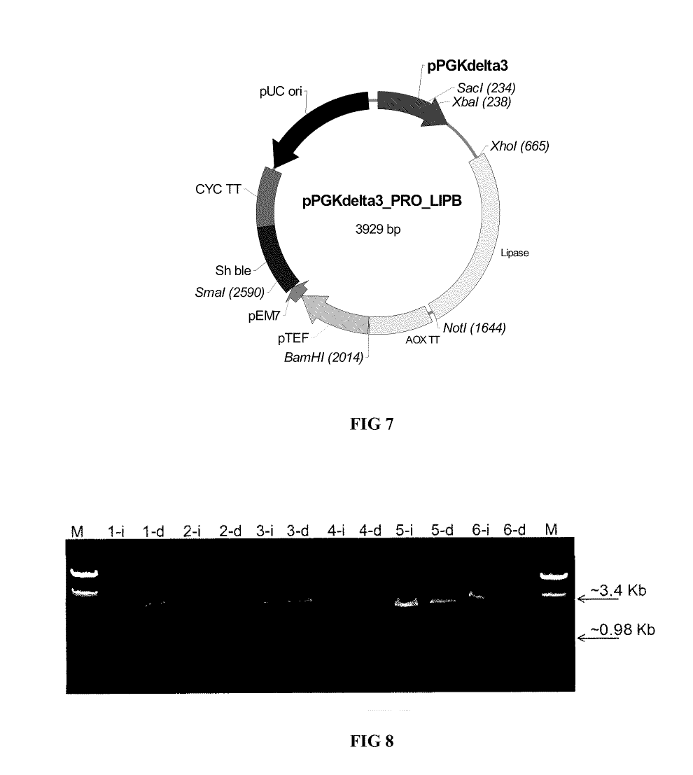

The LipB gene purified from the gel was then cloned into the induced expression vector pPIC9 (FIG. 2) digested with the same restriction enzymes (XhoI and Nod). The resulting vector, called pPIC_LIPB (FIG. 3), was digested with the XhoI and NotI enzymes, thus confirming the correct cloning of the gene (FIG. 4). Cloning of the LipB gene into the constitutive expression vector pPGKΔ3 digested with the XhoI-NotI restriction enzymes was also performed, and the resulting vector was designated pPGKΔ3_LIPB (FIG. 5), which was confirmed by restriction with the same enzymes (FIG. 6). A third construction was carried out, which is a variant of the pPGKΔ3 vector in which the signal peptide was reconstructed with codons optimized for Pichia and the resultant vector was designated pPGKΔ3_PRO_LIPB (FIG. 7), which was also confirmed with the XhoI and NotI enzymes (FIG. 8).

A clone of each construct was selected for large-scale plasmid extraction. Approximately 10 μg of plasmid DNA from each construction was then used for the transformation of P. pastoris.

Transformation in Pichia pastoris

For transformation into P. pastoris, the constitutive expression vectors pPGKΔ3_PRO_LIPB and pPGKΔ3_LIPB were linearized with the restriction enzyme SacI, which cleaves within the sequence of the PGK promoter for the purpose of directing the integration to the locus PGK1 in the genome of P. pastoris. The induced expression vector pPIC_LIPB was linearized with the restriction enzyme DraI for directing the integration to the locus AOX of P. pastoris.

After linearization, the vectors concentrated by precipitation were used for transforming P. pastoris by electroporation. The constitutive vectors were used for transforming the wild-type line X-33 of P. pastoris, the cells having been plated in YPDS medium containing 100 μg/mL zeocin for selection of transformed clones. The induced expression vector was used for transforming the GS115 line (auxotrophic mutant his4) for selection of prototrophic clones His⁺ on plates of minimum medium without histidine. Other exemplary methods for the production of recombinant proteins in yeast are disclosed in Chang et al., J. Agric. Food Chem. 2006, 54, 5831-5838, the published U.S. Patent Applications Nos. 2005/0048649, 2007/0122876 and International PCT Patent Applications WO2010135678 and WO2010099195.

Enzymatic Plate Assay

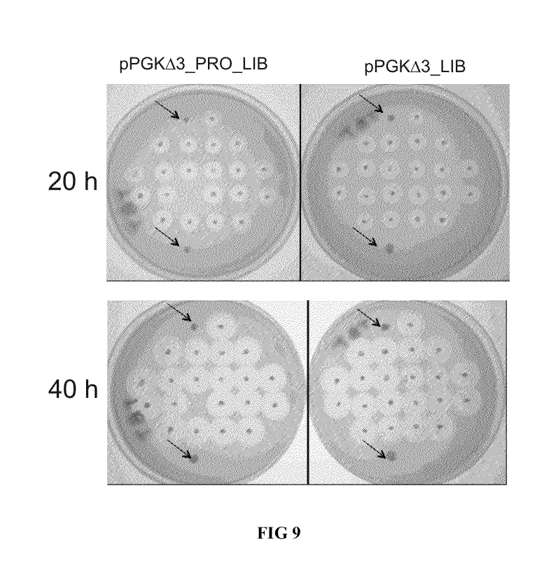

To assess lipase expression, clones resulting from the transformation of P. pastoris were analysed with respect to plate activity. The clones transformed with constitutive expression vectors were tested in YPD agar medium with 1% of tributyrin (emulsified) and following incubation at 28° C. until hydrolysis halos appeared. The pPZα vector that does not contain the lipase gene was used as negative control (FIG. 9). All the transformant clones displayed halos corresponding to recombinant lipase activity.

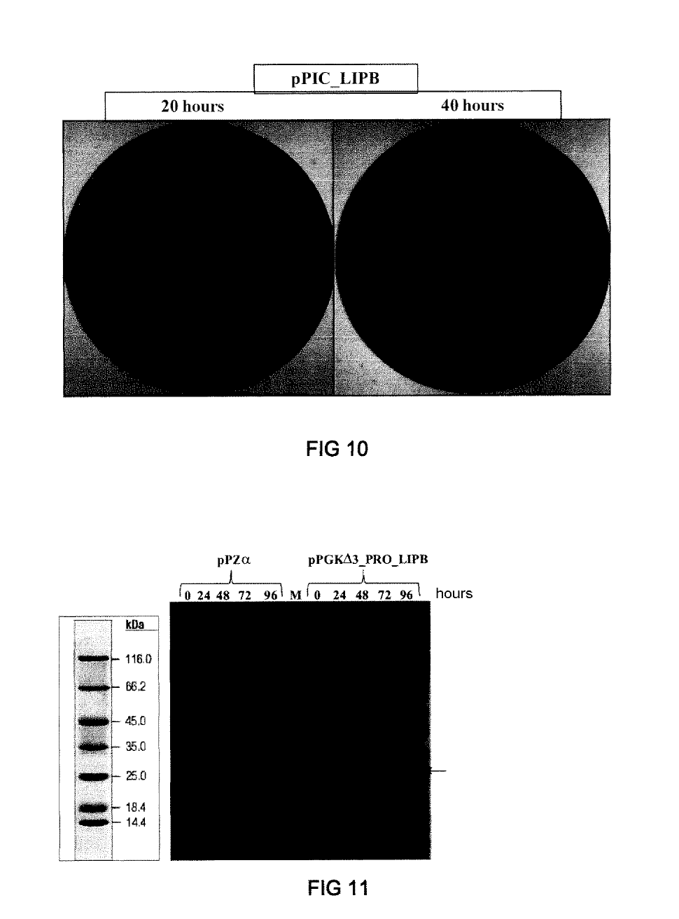

The clones transformed with the induced expression vector were transferred to a plate containing YPM-agar medium (yeast extract 1%, peptone 2% and methanol 0.5%, agar 2%) containing 1% of tributyrin (FIG. 10).

The transformant clones with the constitutive vectors that had larger hydrolysis halos were selected for growth in liquid medium and the transformants with the induced vector with the largest hydrolysis halos were selected and frozen at −80° C. for subsequent analysis.

Quantification of Lipase Activity

Lipase activity was measured using either a spectrophotometric or titrimetric method.

Spectrophotometric Method

Determination of lipase activity was done by incubating 0.05 mL of enzymatic extract with 0.25 mL of a solution containing 2.5 mM of p-nitrophenil palmitate and 2.2 mL of phosphate buffer (25 mM, pH=7.0). Reaction was maintained at 30° C. and absorbance increase (at 412 nm) was monitored on line for 5 minutes. One unit of lipolytic activity corresponds to an amount of enzyme which catalyzes the release of 1.0 μmol of p-nitrophenol per minute under the described conditions. Enzyme activity is expressed as units per volume of liquid culture medium and is referred to in this Application as U (Spectroph.).

Titrimetric Method

The enzyme extract (1 mL) was added to an emulsion (19 mL) of 5% (w/v) olive oil and 5% (w/v) arabic gum in 25 mM phosphate buffer at pH=7.0, and incubated at 35° C. and 200 rpm for 15 min. The reaction was interrupted by the addition of an acetone-ethanol mixture (1:1 v/v), which also promoted the extraction of free fatty acids. These fatty acids were titrated with a pH-stat using 0.04 N NaOH up to a final pH of 11. Reaction blanks were carried out adding the acetone-ethanol mixture prior to the enzyme extract. One lipase unit was defined as the enzyme amount that causes the release of 1 μmol of fatty acids per minute and is referred to in this Application as U (Tritim.)

Analysis of Lipase Expression in Liquid Medium

For growth in liquid medium, a colony isolated from each transformant selected was inoculated in 50 mL of corresponding YPD (initial OD₆₀₀ of 0.09) in a 250 mL Erlenmeyer flask. Culture was carried out in stirred flasks at 28° C. with stirring at 200 rpm for up to 96 hours. At 24-hour intervals, 1 mL aliquots were taken and were stored in 1.5 mL Eppendorff tubes. The supernatant was then tested for the presence of secreted protein using polyacrylamide gel electrophoresis (PAGE). One millilitre of culture supernatant was precipitated with 250 μL of TCA 10% and the pellet was resuspended in 20 μL of sample buffer 2×. The pPZα clone was used as negative control (FIG. 11).

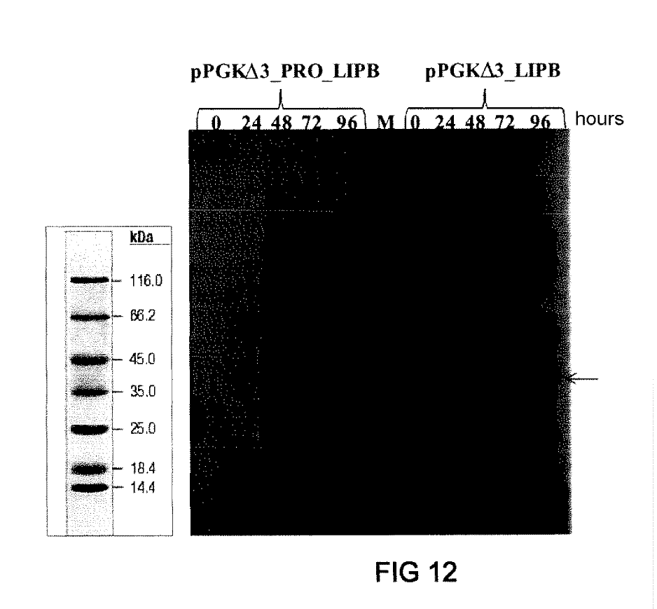

A protein band of ˜37 kDa was observed, which corresponds approximately with the predicted molecular weight of the recombinant lipase. The same band was absent in the negative control. The size predicted for the CALB lipase is 33 kDa. The pPGKΔ3_PRO_LIPB clone, which possesses a signal peptide optimized with preferential codons for P. pastoris, displayed greater expression of CALB than the pPGKΔ3_LIPB clone, as can be seen in FIG. 12.

The results show that the LipB gene corresponding to the CALB lipase of Candida antarctica was expressed very successfully in constitutive and induced form in P. pastoris.

Production of Lipase in a Bioreactor

For preparation of the pre-inoculum, a single colony grown on a plate with YPD solid culture medium, which is widely known and used by persons skilled in the art, was transferred to 10 mL of YPD. The medium was incubated in a rotary agitator at 30° C. with stirring at 250 rpm for 16 hours. An inoculum of 1%-5% was prepared from the pre-inoculum, in 200 mL of YPD medium in a 1 litre halide-treated Erlenmeyer. The medium was incubated in a rotary agitator at 30° C. with stirring at 250 rpm for 12-24 h. After the specified time, the optical density of the inoculum was measured. An initial optical density between 1 and 3 was obtained in the bioreactor. The fermented medium was then centrifuged at 5000 rpm for 5 minutes using a culture volume sufficient for inoculating 1.5 L of YPD medium.

After centrifugation, the supernatant was discarded and the cells were resuspended in sterile medium containing between 1% and 8% (v/v) of glucose, pure glycerol or residual glycerin (from the production of biodiesel) as a substrate. The resuspended cells were then used to inoculate a culture in the bioreactor. Fermentation was carried out at 30° C. with stirring at 300 rpm-800 rpm. The fermentation pH was maintained at 6.0. Under these experimental conditions, the recombinant yeast actively secreted about 12910 U (Tritim.)/L of lipases capable of hydrolysing tributyrin in emulsified medium and about 334 U (Spectroph.)/L of lipases capable of hydrolysing the synthetic substrate p-nitrophenyl palmitate.

Compared with the conventional production of enzymes by native, non-recombinant filamentous fungi, the process time was significantly shorter. Table 1 compares the results for enzyme yield obtained with P. pastoris modified with the optimized synthetic gene from C. antarctica of the present invention, and the yield obtained by the native organism (Penicillium simplicissimum), according to the fermentation process on solid medium described in the Applicant's Brazilian application PI 0703290-0.

TABLE 1

| | Maximum yield | Process time |
|---|---|---|
| Microorganism | (U (Spectroph.)/L.h) | (h) |
| Pichia pastoris | 328 | 24 |
| Penicillium | 242 | 72 |
| simplicissimum | | |

Production of Lipase in a Bioreactor Using Alternative Sources of Carbon

Another aspect examined during the tests relates to the use of glycerin as a substrate. The glycerin used in the experiments was obtained from various sources, for example, soya, castor seed, sweet pine-nut, sunflower, macauba (corozo palm; Acrocomia sclerocarpa) and frying oil.

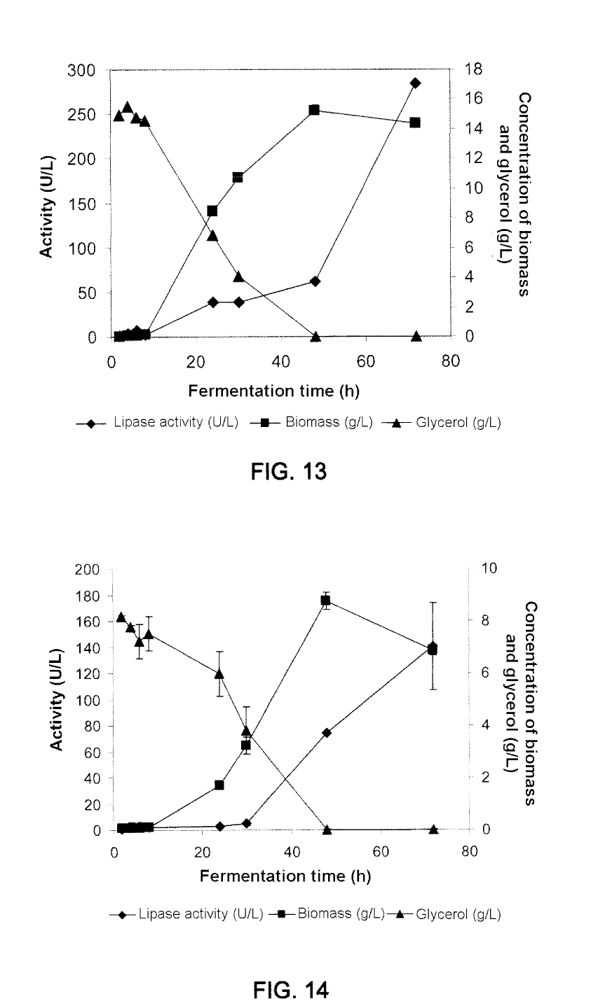

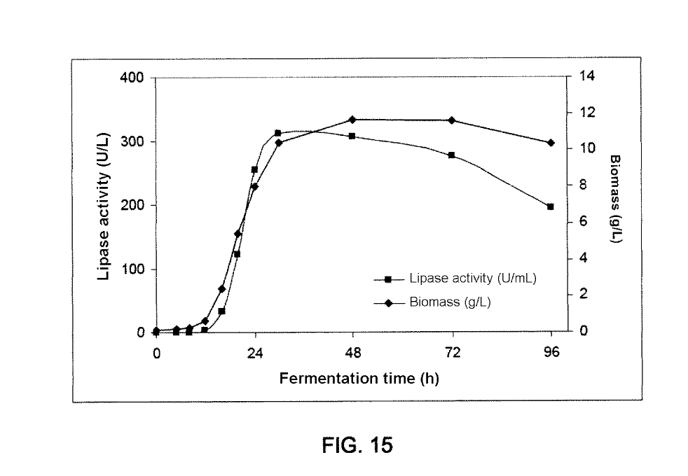

The yeast obtained by genetic modification was capable of growing and producing lipases at significant levels, using clear glycerin (raw glycerin), a residue from production of biodiesel. The results obtained are shown in the graphs in FIGS. 13 and 14.

Table 2 below presents the comparative result for yield (Y_{p/s}) of lipase activity (U(Spectroph.)/amount of added concentration of substrate in grams. The values confirm that the sources obtained from processes for production of biodiesel were metabolized more efficiently than the pure substrate.

TABLE 2

| Source of carbon | Y_{p/s} (U (Spectroph)/g Glycerol) |
|---|---|
| Glycerol P.A. (pure) | 9.2 |
| Clear glycerin from soya | 18.7 |
| Clear glycerin from | 17.5 |
| castor seed | |

The description given thus far of the process for production of lipases by means of construction of synthetic genes and their insertion into the genome of the yeast Pichia pastoris, the object of the present invention, is only to be regarded as one of the possible embodiments, and any particular characteristics mentioned therein are to be understood as being illustrative, only for the purpose of facilitating comprehension. Accordingly, it is not to be regarded as in any way limiting the invention, which is limited to the scope of the claims given hereunder.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

1. A method of producing recombinant lipase in yeast comprising the steps of:
modifying a lipase gene to generate a recombinant lipase gene for optimal expression in the yeast Pichia;
inserting said recombinant lipase gene into an expression vector;
transforming said yeast with said expression vector; and
culturing said transformed yeast to express said recombinant lipase,
wherein said culturing results in the expression of about 334 U (Spectroph.) of recombinant lipase activity per litre of culture media.

2. The method of producing recombinant lipase in yeast according to claim 1, wherein said culturing produces a maximum yield of about 328 Units (Spectroph.) of lipase activity per litre of media per hour.

3. The method of producing recombinant lipase in yeast according to claim 1, wherein said lipase gene is from Candida antarctica, Thermomyces lanuginosus or Pseudomonas cepacia.

4. The method of producing recombinant lipase in yeast according to claim 1, wherein said yeast is Pichia pastoris.

5. The method of producing recombinant lipase in yeast according to claim 1, wherein said recombinant lipase gene has the DNA sequence of SEQ ID NO. 3.

6. The method of producing recombinant lipase in yeast according to claim 1, wherein said recombinant lipase gene has the amino acid sequence of SEQ ID NO. 4.

7. The method of producing recombinant lipase in yeast according to claim 1, wherein said recombinant lipase is secreted efficiently into the culture media.

8. The method of producing recombinant lipase in yeast according to claim 1, wherein said culture media contains glycerin.

9. The method of producing recombinant lipase in yeast according to claim 8, wherein said glycerin is obtained from soya, castor seed, sweet pine-nut, sunflower, macauba or frying oil.

10. The method of producing recombinant lipase in yeast according to claim 8, wherein said glycerin is residual glycerin from the production of biodiesel.

11. The method of producing recombinant lipase in yeast according to claim 10, wherein said culturing in the presence of said residual glycerin increases the yield of recombinant lipase as compared to the yield obtained by culturing in the presence of glycerin.

12. The method of producing recombinant lipase in yeast according to claim 10, wherein said culturing in the presence of said residual glycerin yields about 18.7 U (Spectroph.) of lipase activity per gram of residual glycerin added to the culture media.

13. The method of producing recombinant lipase in yeast according to claim 1, wherein said recombinant yeast is cultured at 30° C. with stirring at 400 rpm and at a pH of about 6.0.

14. The method of producing recombinant lipase in yeast according to claim 1, wherein said transforming said yeast with said expression vector results in the integration of said expression vector into the genome of the host cell.
